(19) United States
(12) Patent Application Publication
(10) Pub. No.: US 2005/0006444 A1
(43) Pub. Date: Jan. 13, 2005

(54) Transparent envelope

(75) Inventor: Raziel Ronen, Tel-Aviv (IL)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(73) Assignee: DEAD SEA MEDICA CO., LTD., South Wales (GB)

(21) Appl. No.: 10/845,617

(22) Filed: May 13, 2004

Related U.S. Application Data
(63) Continuation of application No. PCT/IL02/00899, filed on Nov. 11, 2002.

(30) Foreign Application Priority Data
Nov. 13, 2001 (IL) 146478

(51) Int. Cl. B65D 27/04

(52) U.S. Cl. 229/71; 229/68.1

(57) ABSTRACT

An envelope, formed from a transparent sheet, having edges formed with holes whereby a potentially harmful substance contained therein may be withdrawn via at least one of the holes without tearing the envelope. A method for preventing a potentially harmful substance contained within a transparent envelope from harming bystanders involving visually detecting a potentially harmful substance in the envelope, applying a vacuum to the inside of the envelope, whereby the potentially harmful substance contained therein is discharged through holes into a sealable enclosure for inspection and testing, and discarding the envelope together with its contents if the test reveals that the substance is harmful. In another aspect, the method involves irradiating the envelope with radiation whereby to kill any possible pathogens found within the envelope. In another aspect, the method involves testing the air quality within a mail receptacle, whereby the air quality is indicative of the presence of a harmful substance in an envelope disposed within the receptacle, and secluding a plurality of envelopes if the test reveals a presence of a harmful substance in the receptacle.

### RELATED APPLICATION

This application is a continuation of International application No. PCT/IL02/00899, filed Nov. 11, 2002, the contents of which are here incorporated by reference in their entirety. Applicant claims the benefit of 35 USC 120.

### BACKGROUND OF THE INVENTION

1. Field of the Invention

The present invention relates to the field of office envelopes. More particularly, the present invention relates to a transparent envelope formed with holes whereby a potentially harmful substance contained in the envelope may be withdrawn therefrom via at least one of the holes without tearing the envelope.

2. Prior Art

Personal messages or those types of correspondence that generally require a relatively high level of privacy, such as a bank statement, are usually sent in a sealed opaque envelope. For some business applications, however, a transparent envelope or package is preferred. For example, recipients of a large quantity of shipped merchandise have to return at times articles that have a defect or that were incorrectly shipped. By sending the merchandise, or its documentation, in a transparent envelope, the recipient would be able to examine and, if necessary, return its contents without having to open the envelope. Also, the clerical staff of a large office would be able to expedite the sorting and processing of incoming mail by examining the nature of the correspondence before opening the envelope.

Recently, attempts have been made by terrorists to cause public panic by disseminating biological agents such as anthrax in high-exposure areas and even via the postal service. These agents can be transmitted person-to-person and have the potential of causing a high rate of mortality. Unsuspecting recipients are liable to receive envelopes containing a powdery biological agent. After opening such an envelope, the recipient undergoes a large risk of exposure to the agent. Similarly explosive agents are also liable to be delivered by terrorists to unsuspected recipients by an envelope or package.

Transparent envelopes are well known. For example, International publication WO9950149 teaches a transparent paper product such as an envelope that comprises at least two transparent or translucent surfaces made integral with each other by means of an adhesive. U.S. Pat. No. 6,098,872 discloses an office envelope formed by articulate folds. When more than one sheet of stock is utilized to form the envelope, a transparent sheet may be utilized. US Patent Application 2001/0022317 discloses a flat transparent envelope whose cover sheet and backsheet may be formed of a magnifying material. These publications suffer from the drawback that a potentially harmful substance retained therein may not be withdrawn for inspection without having to tear the envelope.

No speedy and cost-effective means is known, that can be made easily available to the general public, for visually inspecting and withdrawing a portion of the contents of an envelope containing a potentially harmful substance for inspection, without having to tear the envelope.

It is an object of the present invention to provide a transparent envelope.

It is an additional object of the present invention to provide an envelope in which a portion of the contents may be speedily and cost-effectively withdrawn, to allow for the inspection of a potentially harmful substance contained therein.

It is a further object of the present invention to provide an envelope in which a portion of the contents may be withdrawn without having to tear the envelope.

It is a further object of the present invention to provide a method for preventing a potentially harmful substance contained within an envelope from harming bystanders.

Other objects and advantages of the invention will become apparent as the description proceeds.

### SUMMARY OF THE INVENTION

The present invention relates to an envelope formed from a transparent sheet, the edges of said envelope formed with holes whereby a potentially harmful substance contained in said envelope may be withdrawn therefrom via at least one of said holes without tearing said envelope.

The envelope preferably further comprises a front panel, a rear panel, a top flap and side flaps cut from a transparent sheet, each side flap being interspersed between and sealed to the front and rear panels, the top flap capable of being affixed to said rear panel. The envelope is preferably cut and folded from a single sheet.

The holes are preferably formed along the entire periphery of the envelope.

Each hole preferably has a maximum length of 100 mm and ranges from 1 to 100 mm. The spacing between each hole ranges from 1 to 100 mm.

The term “envelope” as referred to herein indicates an external material encompassing a flat or three-dimensional object or objects.

In one preferred embodiment, the envelope is affixed to an opaque protective covering, which is preferably made of paper, the envelope being affixed to an inside face of the protective covering in such a way that the contents of the envelope is not visible upon the folding of said protective covering.

In this embodiment, the rear panel of the envelope preferably faces the inner side of a section of the protective covering, to allow for the opening and closing of the top flap of the envelope. The envelope is preferably releasably affixed to the protective covering. Furthermore, two complementary sections of the protective covering are preferably affixed to each other. The protective covering is preferably provided with a tamper evident design.

The present invention also relates to a method for preventing a potentially harmful substance contained within an envelope from harming bystanders.

In one aspect, the method comprises providing a transparent envelope formed with holes along its periphery, visually detecting a potentially harmful substance in said envelope, applying a vacuum to the inside of the envelope, whereby the potentially harmful substance contained therein is discharged through the holes into a sealable enclosure for inspection and testing, and discarding said envelope together with its contents if the test reveals that the substance is harmful.

The vacuum is preferably applied by means of a vacuum pump which is positioned along each edge of the envelope, whereby the potentially harmful substance is directed into a corresponding discharge conduit, from which it is directed to the sealable enclosure for inspection and testing.

The step of visually detecting a potentially harmful substance may be performed by optically scanning the envelope with intense light, whereby to view minute particles.

In another aspect, the method comprises providing a transparent envelope, visually detecting a potentially harmful substance in said envelope, and irradiating said envelope with radiation whereby to kill any possible pathogens found within the envelope.

The type of radiation is preferably selected from the group of ultraviolet, Gamma rays, X-rays and ultrasound.

With ultraviolet radiation at a wavelength ranging from 50-1000 nanometers and at a distance of up to 10 cm from the envelope, a suitable range of beam power is 1-1,000,000 microwatts at an energy density of 1 cm²/sec.

When Gamma rays are used, a suitable range of dosage is 5-100 kilograys.

When ultrasonic radiation is used, a suitable speed of wave propagation is 1 Mach at a frequency of at least 1 Hz.

In another aspect, the method comprises providing a mail receptacle, providing a plurality of transparent envelopes within said receptacle, each envelope being formed with holes along its periphery, testing the air quality within said receptacle, whereby the air quality is indicative of the presence of a harmful substance in one of said envelopes disposed within the receptacle, and secluding said plurality of envelopes if the test reveals a presence of a harmful substance in the receptacle.

The testing of air quality is preferably tested by means of a covering secured to the mail receptacle, said covering being provided with at least one flexible hose for the influx or discharge of a gas, each hose being equipped with a valve, whereby to apply to release the gas from the mail receptacle.

The air is preferably allowed to circulate throughout the envelopes contained within the mail receptacle and any potentially hazardous substance is entrained in a stream of gas.

In another aspect, the method comprises providing a mail receptacle, providing a plurality of transparent envelopes within said receptacle, each envelope being formed with holes along its periphery, applying a gas to said plurality of envelopes, allowing said gas to circulate within each of said envelopes, whereby said gas kills any pathogens found in said plurality of envelopes.

Ozone being applied at a mass flow rate of 0.001-100 gm/hr or nitrogen being applied at a mass flow rate of 0.001 gm/m³/hr is suitable for this method.

### BRIEF DESCRIPTION OF THE DRAWINGS

In the drawings:

FIG. 1 is a top view of a transparent envelope in one preferred embodiment, according to the present invention;

FIG. 2 is a top view of a sheet used to produce the envelope of FIG. 1;

FIG. 3 is a top view of another preferred embodiment of the present invention, in which the protective covering is shown in an open position;

FIG. 4 is a front view of the embodiment of FIG. 3 in which the protective covering is sealed;

FIG. 5 shows a method of removing the contents of an envelope without tearing; and

FIG. 6 shows another method of removing the contents of an envelope without tearing.

### DETAILED DESCRIPTION OF PREFERRED EMBODIMENTS

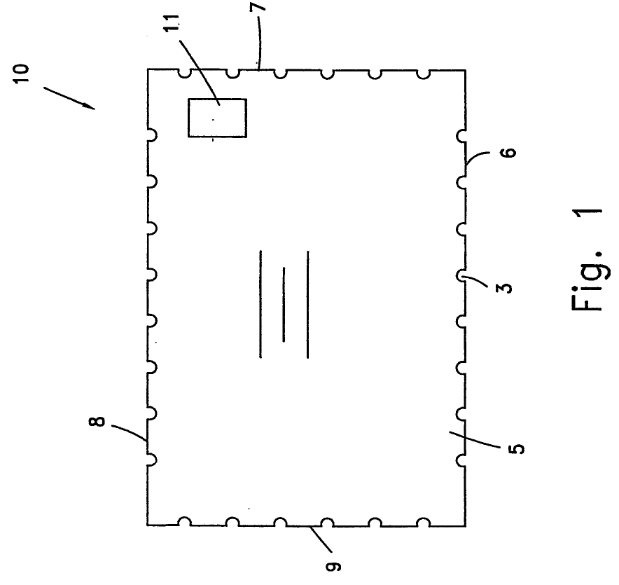

FIG. 1 illustrates one preferred embodiment of the present invention in which a conventional envelope, generally designated by 10, is made from a singular transparent and water impermeable sheet 5, such as polyethylene, polypropylene, nylon, polyamide, polymers, or from any other material that is adapted for the production of a transparent web. A plurality of semicircular holes 3, or of any other preferred shape, are formed along the four edges 6, 7, 8, 9 of the envelope, such that each edge is not rectilinear due to the formation of the holes. The holes may be formed by a punch or press, and alternatively may be reinforced. Holes 3 allow for the passage of air into the enclosure defined by envelope 10, and consequently enable any powder contained therein to be released to the surroundings. Each hole has a maximum length of 100 mm, and preferably ranges from 1 to 100 mm. The spacing between each hole ranges from 1 to 100 mm. Stamp 11 is applied to the envelope by roughening an appropriate location on sheet 5 or by applying an adhesive. A mailing label (not shown) may be similarly applied.

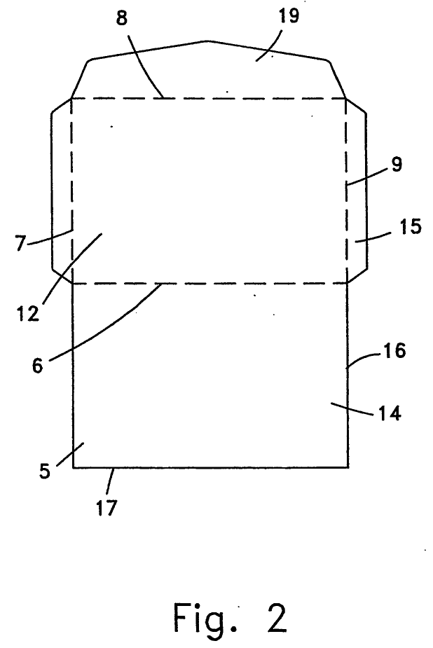

Sheet 5, as shown in FIG. 2, comprises two rectangular sections corresponding to front panel 12 and rear panel 14, two side flaps 15 and a top flap 19. The edges of the envelope, which are indicated by the dotted lines, are formed by folding the envelope in the following way. Edge 6 results by folding sheet 5 at the connecting points between each side flap 15 and corresponding side 16 of sheet 5, whereby bottom edge 17 of sheet 5 is aligned with top edge 8 of the envelope. Edges 7 and 9 are formed by folding side flaps 15 inward, such that side flaps 15 are interspersed between front panel 12 and rear panel 14. Edge 8 is formed by folding top flap 19 over rear panel 14. Side flaps 15 are heat-sealed to rear panel 14. Top flap 19 is affixed to rear panel 14 by a water soluble glue, as is well known to those skilled in the art, by a staple, or by any other suitable means of affixing. The transparent envelope may also be made in any desired shape, and may not necessarily made from a single sheet.

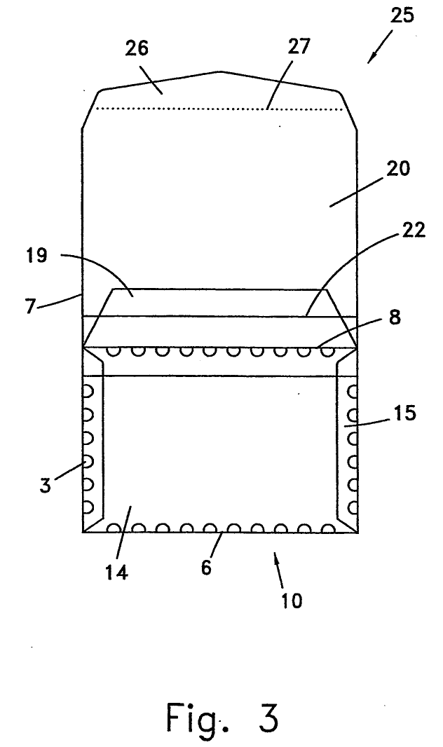

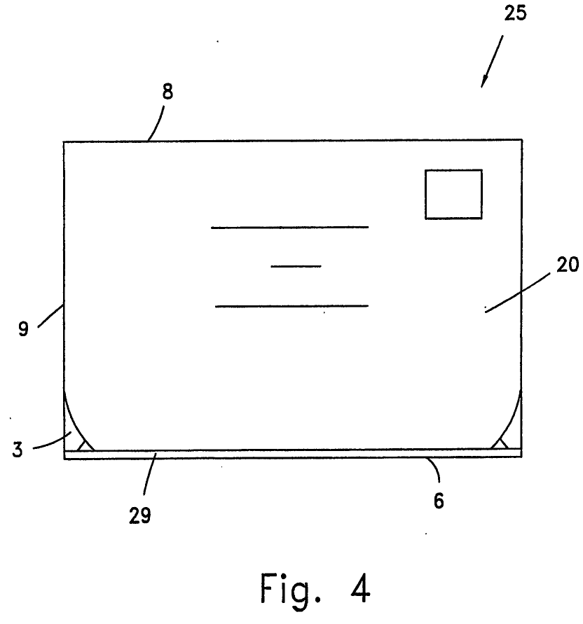

FIG. 3 illustrates another preferred embodiment of the present invention in which transparent envelope 10 is affixed to opaque protective covering 25, preferably made from paper. Upon the folding of protective covering 25 at crease 22 (FIG. 4), the contents of the envelope is not visible. Transparent envelope 10 is adhered to an inside face of the protective covering with at least one strip of two-sided adhesion, yet is capable of being detached without tearing from the protective covering for subsequent inspection. Bottom edge 6 of envelope 10 essentially corresponds to the bottom edge of protective covering 25, while top edge 8 is downwardly disposed, i.e. in the direction of bottom edge 6, with respect to crease 22 to facilitate the folding of the protective covering. Rear panel of the envelope preferably faces the inner side of section 20 of the protective covering, to allow for the opening and closing of top flap 19 of the envelope. The provision of such a protective covering allows for the quick processing of envelopes, e.g. by a conveyor belt. The protective covering also advantageously prevents a corner of one envelope to be engaged by one of the holes. If a hole inadvertently retains one of the envelope corners, the rate of mail processing would be restricted, and possibly an envelope may be sent to an incorrect location.

Protective covering 25 is preferably provided with a decorative design indicating that a transparent envelope in contained therein, in order to prevent tampering with the envelope. The two complementary sections of protective covering 25 are affixed to each other, e.g. by flap 26, or by any other affixing means. Flap 26 may be releasably affixed, such as by a resealable seal or by a slit formed in one of protective covering sections which is suitable for receiving and securing the flap. The side edges of the protective covering are not sealable, to prevent any surreptitious holding of a potentially harmful substance in any portion of the protective covering. As shown in FIG. 4, protective covering 25 is preferably provided with tamper-proof strip 29. Accordingly, if a harmful substance is inserted into envelope 10, such as through holes 3, strip 29 will be torn and the protective covering will immediately identify that tampering has occurred.

Upon detection of a potentially harmful substance within an envelope, some of the following methods may be implemented to prevent harm to bystanders:

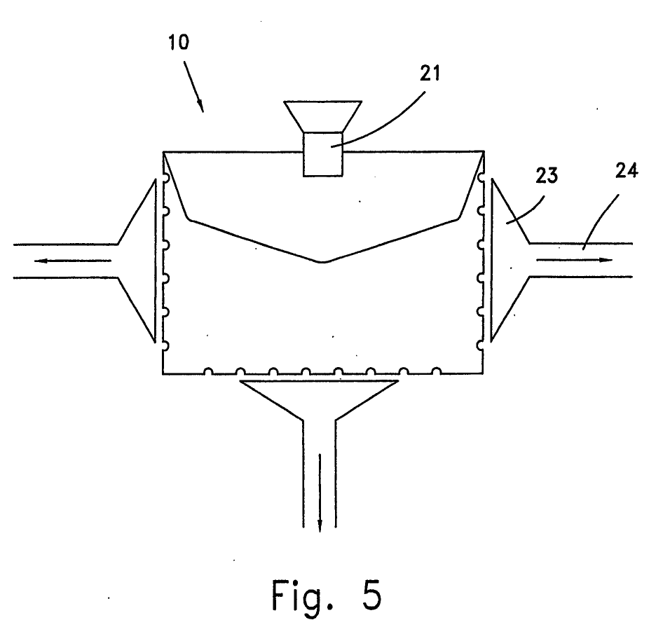

Applying a vacuum ranging from 0.001 to 1000 millibar to the inside of the envelope, e.g. by means of a low vacuum pump, whereby the potentially harmful substance contained therein is discharged through the holes into a sealable enclosure for inspection and testing. As shown in FIG. 5, envelope 10 is held by clip 21, or by any other fixation device. A vacuum pump 23 is positioned along each edge of the envelope, so that the potentially harmful substance is directed into a corresponding discharge conduit 24, from which it is directed to a sealable enclosure for inspection and testing. If a harmful substance is discovered, the envelope will be discarded.

Irradiating the envelope with radiation at a predetermined dosage to thereby kill any possible pathogens found within the envelope. When ultraviolet radiation is used, at a wavelength ranging from 50-1000 nanometers and at a distance of up to 10 cm from the envelope, a suitable range of beam power is 1-1,000,000 microwatts at an energy density of 1 cm²/sec. When Gamma rays are used, a suitable range of dosage is 5-100 kilograys, for all suitable frequencies. When X-rays are used, a suitable beam power is selected which is sufficient for the maximum volume of harmful substance that is to be treated. When ultrasonic radiation is used, a speed of wave propagation of 1 Mach at a frequency of at least 1 Hz is suitable.

Applying a gas to large batches of mail before distribution to thereby kill any possible pathogens found within the envelopes. The application of ozone at a mass flow rate of 0.001-100 gm/hr is sufficient to cause oxidation of and to instantly kill any possible pathogens found within the envelope. The application of nitrogen at a mass flow rate of 0.001 gm/m³/hr is sufficient to prevent the influx of air and consequently to choke any possible pathogens found within the envelope after a predetermined period of time.

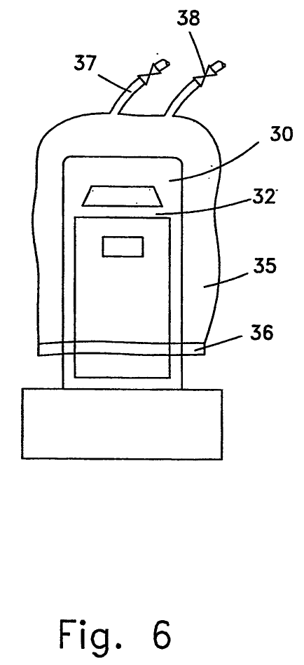

Testing the air quality within a mailbox, or any other enclosure containing a multiplicity of envelopes formed with holes. As shown in FIG. 6, flexible and transparent covering 35, which may be a plastic bag, is provided to encompass mailbox 30. Covering 35 is releasably secured to the mailbox by securing means 36, which may be an elastic or Velcro strip. Covering 35 is provided at the top thereof with at least one flexible hose 37. Each hose 37 is equipped with valve 38, e.g. a check or ball valve, to allow for the influx or discharge of a gas. Pressurized air may be introduced via valve 38 and slit 32 into the interior of mailbox 30. The air is allowed to circulate throughout the envelopes contained within the mailbox, and any potentially hazardous substance is entrained in the stream of air. After a predetermined period of time, air is released from the mailbox via valve 38 for testing. As a result, foreign matter may be extracted from the envelope or interior of the mailbox without having to remove the envelope itself.

This method may be employed to introduce a gas for sanitizing the interior of the mailbox. Furthermore, ozone or nitrogen, with the aforementioned flow rates, may be introduced into the mailbox for treatment of possible pathogens located therein.

Optically scanning each envelope individually. By viewing the contents of an envelope with intense light, minute particles may be seen, which ordinarily would not have been noticed if viewed with normal illumination.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

1. An envelope formed from a transparent sheet, the edges of said envelope formed with holes whereby a potentially harmful substance contained in said envelope may be withdrawn therefrom via at least one of said holes without tearing said envelope.

2. Envelope according to claim 1, further comprising a front panel, a rear panel, a top flap and side flaps cut from a transparent sheet, each side flap being interspersed between and sealed to the front and rear panels, the top flap capable of being affixed to said rear panel

3. Envelope according to claim 2, wherein the envelope is cut and folded from a single sheet.

4. Envelope according to claim 1, wherein the holes are formed along the entire periphery of the envelope.

5. Envelope according to claim 1, wherein each hole has a maximum length of 100 mm and ranges from 1 to 100 mm.

6. Envelope according to claim 1, wherein the spacing between each hole ranges from 1 to 100 mm.

7. Envelope according to claim 1, wherein the envelope is affixed to an opaque protective covering, the envelope being affixed to an inside face of the protective covering in such a way that the contents of the envelope is not visible upon the folding of said protective covering.

8. Envelope according to claim 7, wherein the rear panel of the envelope faces the inner side of a section of the protective covering, to allow for the opening and closing of the top flap of the envelope.

9. Envelope according to claim 7, wherein the envelope is releasably affixed to the protective covering.

10. Envelope according to claim 7, wherein two complementary sections of the protective covering are affixed to each other.

11. Envelope according to claim 7, wherein the protective covering is provided with a tamper evident design.

12. Method for preventing a potentially harmful substance contained within an envelope from harming bystanders, comprising:
providing a transparent envelope formed with holes along its periphery, visually detecting a potentially harmful substance in said envelope, applying a vacuum to the inside of the envelope, whereby the potentially harmful substance contained therein is discharged through the holes into a sealable enclosure for inspection and testing, and discarding said envelope together with its contents if the test reveals that the substance is harmful.

13. Method of claim 12, wherein the vacuum is applied by means of a vacuum pump which is positioned along each edge of the envelope, whereby the potentially harmful substance is directed into a corresponding discharge conduit, from which it is directed to the sealable enclosure for inspection and testing.

14. Method of claim 12, wherein the step of visually detecting a potentially harmful substance is performed by optically scanning the envelope with intense light, whereby to view minute particles.

15. Method for preventing a potentially harmful substance contained within an envelope from harming bystanders, comprising:
providing a transparent envelope, visually detecting a potentially harmful substance in said envelope, and irradiating said envelope with radiation whereby to kill any possible pathogens found within the envelope.

16. Method of claim 15, wherein the type of radiation is selected from the group of ultraviolet, Gamma rays, X-rays and ultrasound.

17. Method of claim 16, wherein the type of radiation is ultraviolet radiation at a wavelength ranging from 50-1000 nanometers and at a distance of up to 10 cm from the envelope, a suitable range of beam power being 1-1,000,000 microwatts at an energy density of 1 cm²/sec.

18. Method of claim 16, wherein the type of radiation is Gamma rays, a suitable range of dosage being 5-100 kilograys.

19. Method of claim 16, wherein the type of radiation is ultrasonic radiation, a suitable speed of wave propagation being 1 Mach at a frequency of at least 1 Hz.

20. Method for preventing a potentially harmful substance contained within an envelope from harming bystanders, comprising:
providing a mail receptacle, providing a plurality of transparent envelopes within said receptacle, each envelope being formed with holes along its periphery, applying a gas to said plurality of envelopes, allowing said gas to circulate within each of said envelopes, whereby said gas kills any pathogens found in said plurality of envelopes.

21. Method of claim 20, wherein the gas is ozone, ozone being applied at a mass flow rate of 0.001-100 gm/hr.

22. Method of claim 20, wherein the gas is nitrogen, nitrogen being applied at a mass flow rate of 0.001 gm/m³/hr.

23. Method for preventing a potentially harmful substance contained within an envelope from harming bystanders, comprising:
providing a mail receptacle, providing a plurality of transparent envelopes within said receptacle, each envelope being formed with holes along its periphery, testing the air quality within said receptacle, whereby the air quality is indicative of the presence of a harmful substance in one of said envelopes disposed within the receptacle, and secluding said plurality of envelopes if the test reveals a presence of a harmful substance in the receptacle.

24. Method of claim 23, wherein the testing of air quality is tested by means of a covering secured to the mail receptacle, said covering being provided with at least one flexible hose for the influx or discharge of a gas, each hose being equipped with a valve, whereby to apply to release the gas from the mail receptacle.

25. Method of claim 24, wherein the air is allowed to circulate throughout the envelopes contained within the mail receptacle and any potentially hazardous substance is entrained in a stream of gas.
